(19) United States
(12) Patent Application Publication
(10) Pub. No.: US 2005/0032205 A1
(43) Pub. Date: Feb. 10, 2005

(54) In vitro cell culture employing a fibrin network in a flexible gas permeable container

(76) Inventors: Sidney T. Smith, Lake Forest, IL (US); Stephen Lee Smith, Arlington Heights, IL (US); James P. Diorio, Antioch, IL (US); Susan K. Young, Gurnee, IL (US); David V. Bacehowski, Wildwood, IL (US); T. Michael Dennehey, Arlington Heights, IL (US)

Correspondence Address: BAXTER HEALTHCARE CORPORATION;RENAL DIVISION, 1 BAXTER PARKWAY, DF3-3E, DEERFIELD, IL, 60015, US

(21) Appl. No.: 10/634,663

(22) Filed: Aug. 5, 2003

(51) Int. Cl. C12M 3/04; C12N 5/02

(52) U.S. Cl. 435/297.5; 435/299.2; 435/401; 435/402

(57) ABSTRACT

This invention relates to in vitro cell culture employing a fibrin network in a flexible gas permeable container. Specifically, the invention is directed to a cell culture container comprising a flexible, gas permeable material with fibrin matrix which is conducive to the culture of anchorage dependent cells, and the container is suitable for use in closed system in vitro cell culture. The gas permeability of the container is sufficient to permit cellular respiration.

### CROSS-REFERENCE TO RELATED APPLICATIONS

Not Applicable.

### FEDERALLY SPONSORED RESEARCH OR DEVELOPMENT

Not Applicable.

### BACKGROUND OF THE INVENTION

### TECHNICAL FIELD

This invention relates to in vitro cell culture employing a fibrin network in a flexible gas permeable container. Specifically, the invention is directed to a cell culture container comprising a flexible, gas permeable material with fibrin matrix which is conducive to the culture of anchorage dependent cells, and the container is suitable for use in closed system in vitro cell culture.

### BACKGROUND OF THE INVENTION

There are two major types of cells grown in vitro: suspension cells (anchorage-independent cells) and adherent cells (anchorage-dependent cells). Suspension or anchorage-independent cells can multiply in vitro without being attached to a surface. In contrast, adherent cells require attachment to a surface in order to grow in vitro. Additionally, some non-adherent cells grow best on a surface that promotes adherent cell growth.

It is known to grow adherent cells in vitro in polystyrene flasks. Polystyrene is the most common type of plastic used in the manufacture of rigid, gas impermeable cell culture flasks or plates. It is thought that polystyrene promotes the growth of adherent cells because of its ability to maintain electrostatic charges on its surface which attract oppositely charged proteins on the cell surfaces. However, to date, the available polystyrene culture containers have been of the rigid flask or plate type because polystyrene is known in the art as a rigid, gas-impermeable plastic.

Cells are commonly cultured in a growth medium within polystyrene or other containers placed in enclosed incubators. In addition to providing a limited degree of isolation from microbial contamination, the incubators maintain a constant temperature, usually 37° C., and a constant gas mixture. The gas mixture may be optimized for a given cell type, and be controlled for at least two parameters: (1) partial pressure of oxygen (pO₂) to serve the aerobic needs of the cells, and (2) partial pressure of carbon dioxide (pCO₂) to maintain the pH of the growth medium. Since the known types of rigid cell culture containers are gas impermeable, their lids or caps are not sealed onto the containers. Rather, they are offset sufficiently to allow gas exchange through a gap or vent between the cap and the container. Such a container is disadvantageous for clinical uses because the vent might allow contamination of the culture or lead to accidents involving biohazardous agents. Cultured tissues grown in vented vessels are unsuitable for transplantation and therapeutic applications.

In addition to polystyrene flasks, others have constructed flexible, breathable containers for containing adherent cells to be grown in vitro. (See U.S. Pat. Nos. 4,588,401; 4,496,361; 4,222,379; and 4,140,162). The commonly assigned U.S. Pat. No. 4,939,151 provides a gas permeable bag with at least one access port. This allows for a closed system (i.e., one without a vent). The bag disclosed in the '151 patent is constructed from two side walls. The first side wall is made of ethylene-vinyl acetate (“EVA”) which may be positively or negatively charged. The second side wall is constructed from a gas permeable film such as ethylene-vinyl acetate or a polyolefin. The first side wall is sealed to the second side wall along their edges. While EVA can hold an electrostatic charge, the charge has the undesirable tendency to decay over time. Eventually, the decay of the charge on EVA will render the container ineffective for growing adherent cells. Rigid styrene flasks with an electrostatic charge are known, and show less of a tendency to lose charge over time.

It has been found that the cell growth rate within a sealed container may be influenced by the gas permeability characteristics of the container walls. The optimal gas requirements, however, vary by cell type and over the culture period. Thus, it is desirable to be able to adjust the gas permeability of the container. The polystyrene flask, and the flexible flask which is entirely constructed from a monofilm, do not provide for such adjustability.

Another commonly assigned U.S. Pat. No. 5,935,847 provides a gas permeable container constructed from a multilayer, flexible, gas permeable film comprising an inner cell growth surface and a polymeric layer. The cell growth layer is composed of polystyrene and the polymeric layer comprises a multiple component polymer alloy blend containing styrene and diene copolymers and/or styrene and alpha-olefin copolymers.

The container in the '847 patent is used for the in vitro culture of adherent and/or non-adherent cells. The gas permeability of the container may be adjusted to best match the requirements of the cell being cultured by varying the material and thickness of the polymeric layer. However, there is a need for primary biocompatibility from the container. This requirement for biocompatibility can be obtained by incorporating a fibrin matrix in a gas permeable container, such as the containers disclosed in the '151 and '847 patents. A fibrin matrix having specific conformation and three dimensional characteristics can create a framework for the culture of cells, tissues and perhaps portions of organs. The cells adhere to and embed in the matrix, so that the spatial characteristics of the matrix can be conferred upon the tissue growing thereon.

Fibrin matrices are well-known in the art for use in hemostasis, tissue sealing and wound healing. Fibrin sealants/glues have been commercially available for more than a decade for these purposes. Fibrin sealants/glues mimic the last step of the coagulation cascade and are usually commercialized as kits having two main components. The first component is a solution comprising fibrinogen and factor XIII, while the second component is a thrombin-calcium solution. After mixing of components, the fibrinogen is proteolytically cleaved by thrombin and thus converted into fibrin monomers. In the presence of calcium, Factor XIII is also cleaved by thrombin into its activated form FXIIIa. FXIIIa cross-links the fibrin monomers to a three-dimensional network to form a fibrin matrix.

The ability of fibrin matrix to support cellular or tissue growth is known in the art. For example, U.S. Pat. Nos. 5,272,074 and 5,324,647 disclose methods for coating a surface of a polymeric material such as polyethylene, polyethyleneterephthalate or expanded polytetrafluoroethylene with fibrin. The fibrin-coated surfaces provide substrates for the growth of endothelial cells, prosthetic devices (including vascular grafts) having reduced thrombogenicity, and test systems for the study of thrombogenesis and fibrinolysis. U.S. Pat. No. 5,912,177 discloses a system for selectively immobilizing and culturing stem cells onto the inner surface of a flexible container. The system comprises a closed container formed of a flexible plastic material which is permeable to carbon dioxide and oxygen. The container includes a substrate having a coating disposing a fibrin matrix. The system requires a substance capable of binding to the fibrin matrix and having an RGD amino acid sequence for binding to the stem cells.

By incorporating a fibrin matrix in a flexible cell culture container, the fibrin matrix lessens the functional biocompatibility requirements of the materials from which the container is fabricated. By transferring the biocompatibility requirement of the culture from the container to the fibrin matrix, the material selection of the container can focus on other attributes, such as gas permeability, optical clarity, and material strength. The container is well suited for applications involving therapeutic transplantation of cultured cells. The container is permeable to gases, but not vented, thereby maintaining an environment free of contaminants during cell culture and processing. The fibrin matrix provides an environment conducive to the adherence and proliferation of certain mammalian cell types. Although it is known that “anchorage dependent” or “adherent” cells can be cultured in fibrin matrices incorporated into rigid styrene T-flasks, cell culture techniques employing a fibrin matrix in a flexible, gas permeable container have not been pursued.

### SUMMARY OF THE INVENTION

The present invention provides a flexible, gas permeable cell culture container with a fibrin matrix suitable for closed system in vitro cell culture. The container is most suitable for culturing anchorage dependent mammalian cells for expansion and transplantation.

The container comprises a supportive container with a fibrin matrix. The supportive container has a first side wall connected to a portion of a second side wall along a peripheral seal to define a containment area. Each side wall has an interior surface. The first side wall of the supportive container is constructed from a flexible, gas permeable material selected from the group consisting of polymeric material, paper, and fabric. The second side wall is constructed either from a flexible, gas permeable material which may be the same or different from the material of the first side wall, or from a flexible, non-gas permeable material selected from the group consisting of polymeric material, paper, fabric, and metal foil. The gas permeability of the container is sufficient to permit cellular respiration. A portion of the interior surface of one of the side walls is covered by a fibrin matrix to provide an environment conducive to adherent cell proliferation and maturation.

The flexible, gas permeable material is preferably a polymeric material. Suitable polymeric materials include ethylene vinyl acetate copolymers, polyolefins, polyamides, polyesters, styrene and hydrocarbon copolymers, and fluorocarbon elastomers (FEP). A preferred polymeric material is ethylene vinyl acetate copolymer (EVA). Another preferred polymeric material is a multiple-component polymer blend. Preferably, at least one of the components of the multiple-component polymer blend is a styrene and hydrocarbon copolymer.

The fibrin matrix should cover at least a portion of the interior surface of one of the side walls. Preferably, the fibrin matrix should cover a substantial portion of the interior surface of one of the side walls. The fibrin matrix is preferably three-dimensional having pore sizes of from about 0.5 μm to about 5.0 μm.

The present invention also provides a method for culturing cells in a closed system in vitro cell culture using the flexible, gas permeable container with a fibrin matrix in accordance with the present invention. By employing a closed system, the invention is well suited for applications involving therapeutic transplantation of cultured cells.

One aspect of the present invention is to transfer the adherent cell culture growth performance from entire dependence on container and material attributes to physical properties of the fibrin matrix to permit greater control of cell culture parameters. By independently varying the characteristics of the fibrin matrix and gas permeability of the container material, cell culture conditions can be optimized for a variety of cell lines.

Another aspect of the present invention is to practice “closed system” cell culture, lending the procedure to therapeutic applications. The container can potentially be used to generate formed tissue, not just individual cells and small aggregates.

Another aspect of the present invention is to more readily accommodate other container attributes such as clarity, strength, or choice of material.

These and other aspects and attributes of the present invention will be discussed with reference to the following drawings and accompanying specification.

### BRIEF DESCRIPTION OF THE DRAWINGS

FIG. 1a is a partial cross-sectional view of the flexible, gas permeable supporting container without the fibrin matrix;

FIG. 1b is a partial cross-sectional view of the flexible, gas permeable cell culture container with the fibrin matrix covering the interior of the side wall;

FIG. 2 is a partial cross-sectional view of a multilayer flexible, gas permeable structure for constructing the supporting container. This embodiment is a two-layer structure;

FIG. 3 is a partial cross-sectional view of a two-layer flexible, gas permeable structure having a first polystyrene layer and a second polymeric layer;

FIG. 4 is a perspective view of a flexible, gas permeable container of the present invention having a fill port and access ports;

FIG. 5a is a plan view of an embodiment of the support container with a fitment having one fill port and two access ports;

FIG. 5b is a side elevational view of the container of FIG. 5a;

FIG. 6 is a perspective view of a fibrin delivery device which can be adapted to loading the fibrin into the inner surface of a side wall of the supporting container through a fill port;

FIG. 7 is a partial perspective view of a fibrin delivery device of FIG. 6 docking at a fill port of the supporting container of FIG. 4 for delivering the fibrin matrix into the supporting container;

FIG. 8 is micrograph of Scanning Electron Microscopy of a PL269 container with fibrin matrix showing the fibrin matrix with adherent cells on the container's interior surface;

FIG. 9 is a photomicrograph of a culture of “anchorage dependent” cells in a polystyrene flask without a fibrin matrix after 4 days of culture;

FIG. 10 is a photomicrograph of a culture of “anchorage dependent” cells in a PL269 container with fibrin matrix after 4 days of culture; and

FIG. 11is a photomicrograph of a culture of “anchorage dependent” cells in a PL269 container without the fibrin matrix after 4 days of culture.

### DETAILED DESCRIPTION OF THE INVENTION

While this invention is susceptible of embodiments in many different forms, there is shown in the drawings and will herein be described in detail a preferred embodiment of the invention with the understanding that the present disclosure is to be considered as an exemplification of the principles of the invention and is not intended to limit the broad aspect of the invention to the embodiments illustrated.

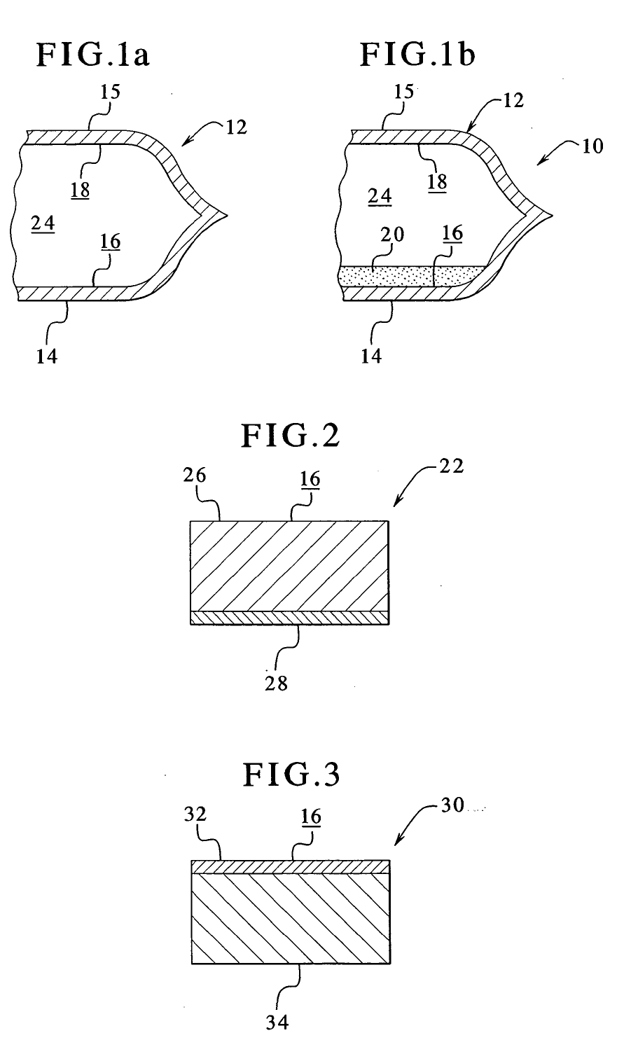

Referring to the figures, FIG. 1b is a partial cross-sectional view of a closed system cell culture container 10 of the present invention comprising a supporting container 12 constructed from flexible, gas permeable materials and a fibrin matrix 20 incorporated into the supporting container.

I. Materials

The walls of the supporting container 12 can be constructed from any material that exhibits sufficient properties of optical clarity, gas permeability, and flexibility. The supporting container 12 should be flexible and should have sufficient gas permeabilities for carbon dioxide and oxygen to support cellular respiration during the culture. It is preferred that the supporting container 12 be optically transparent to allow observation of the cells during culture. The biocompatibility of the container 10 is provided or supplemented by the fibrin matrix 20 within the supporting container 12 and not necessarily from the materials in constructing the supporting container 12.

A preferred material for constructing the supporting container 12 is a polymeric material. Suitable polymeric materials include, but are not limited to, ethylene vinyl acetate copolymers (EVA), polyolefins, polyamides, polyesters, styrene and hydrocarbon copolymers, and fluorocarbon elastomers.

A preferred polymeric material is polyethylene vinyl acetate copolymers (EVA). The use of EVA for flexible cell culture containers is disclosed in the commonly assigned U.S. Pat. No. 4,939,151, which is hereby incorporated by reference and made a part hereof. Containers constructed from EVA are generally transparent, flexible, and gas permeable. In a preferred from of the invention, the vinyl acetate is present in an amount by weight of greater than 18% of the ethylene vinyl acetate copolymer.

Another preferred polymeric material is a multiple-component polymer blend. Examples of multiple-component polymer blends are disclosed in the commonly assigned U.S. Pat. No. 5,935,847, which is hereby incorporated by reference and made a part hereof. Preferably, at least one of the components of the multi-component polymer blend is a styrene and hydrocarbon copolymer. In another preferred embodiment, the polymer alloy blend includes an ethylene vinyl acetate.

In one embodiment, the polymer alloy blend has three components. Preferably, a first component is a styrene-ethylene-butene-styrene block copolymer, a second component is ethylene vinyl acetate, and a third component is polypropylene. The styrene-ethylene-butene-styrene block copolymer preferably constitutes from about 40% to about 85% by weight of the polymer alloy, the ethylene vinyl acetate constitutes from about 0% to about 40% by weight of the polymer alloy, and the polypropylene constitutes from about 10% to about 40% by weight of the polymer alloy.

In another embodiment, the polymer alloy blend is a four component polymer alloy blend. Preferably, a first component is a polypropylene, a second component is selected from the group consisting essentially of an ultra low density polyethylene and polybutene-1, a third component of a dimer fatty acid polyamide, and a fourth component of a styrene-ethylene-butene-styrene block copolymer. In a preferred embodiment, the first component constitutes within the range of from about 30% to about 60% by weight of the polymer alloy, the second component constitutes within the range of from about 25% to about 50% by weight of the polymer alloy, the third component constitutes within the range of from about 5% to about 40% by weight of the polymer alloy, and the fourth component constitutes from about 5% to about 40% by weight of the polymer alloy.

The supporting container 12 can also be constructed from suitable flexible, gas permeable non-polymeric materials such as paper and fabric.

In one embodiment of the invention, part of the supporting container 12 may be constructed from flexible but non-gas permeable materials which include, but are not limited to, polymeric materials, paper, fabric, and metal foil.

The supporting container 12 can be constructed from monolayer or multilayer structures made from the materials described above. One of the main advantages of using multilayer structures is that the materials and dimensions of the included layers as well as the overall structures provide numerous alternatives and choices for achieving the appropriate physical properties such as gas permeabilities and flexibility to meet the various requirements of specific cells.

In a preferred form of the invention, a multilayer structure is made from EVA. The EVA structure 22 shown in FIG. 2 is a two-layer structure. In this structure, the inner layer 26 is composed of EVA with preferably a vinyl acetate content of greater than 18% by weight of the copolymer. Adhering to the inner layer 26 is a higher modulus EVA skin layer 28 having a vinyl acetate content of preferably less than 18%, and more preferably about 9%, by weight of the copolymer. An optional tie layer between the inner layer 26 and the skin layer 28 can be included in the two-layer EVA structure. The tie layer providing adhesive compatibility between the first and second layers. Preferably, the tie layer is composed of a gas permeable olefin. A preferred gas permeable olefin is an ethylene polymer containing vinyl acetate preferably within the range of 16%-32% by weight, and more preferably 28% by weight. Although it is preferred that both the inner layer 26 and the skin layer 28 be composed of EVA, other polymeric material such polyolefins, polyamides, polyesters and the like can be selected to form the skin layer 28. The layers in this multilayer structure are generally coextruded. An example of an EVA structure suitable for the present invention is available from Baxter Healthcare Corporation (Deerfield, Ill.), under the product designation of PL269®.

In another preferred embodiment, the multilayer structure is made of polymeric material as disclosed in the commonly assigned U.S. Pat. No. 5,935,847. A cross-sectional view of a preferred multilayer structure is shown in FIG. 3. The multilayer structure 30 comprises an ultra thin polystyrene layer 32 having a thickness from about 0.0001 inches to about 0.0010 inches. One side of the polystyrene layer 32 forms the interior surface 16 or 18 of the side wall 14 or 15, respectively, of the support container. A second polymeric layer 34 adhered to the other side of the polystyrene layer 32 is made of a polymeric material having a thickness of preferably from about 0.004 inches to about 0.025 inches. Suitable polymeric materials for the layer 34 includes, but are not limited to, polyolefins, polyamides, polyesters, and styrene and hydrocarbon copolymers. In a preferred embodiment, the polymeric material of layer 34 is a multiple-component polymer blend. In another preferred embodiment, at least one of the components of the multi-component polymer blend is a styrene and hydrocarbon copolymer. Optionally, the multilayer structure 30 may have an additional skin layer as described previously.

An example of the multilayer structure in FIG. 3 is available from Baxter Healthcare Corporation (Deerfield, Ill.) under the product designation of PL-2417®.

II. Supporting Container

FIG. 1a is a cross-sectional view of an embodiment of the flexible, gas permeable supporting container without a fibrin matrix. The supporting container 12 is preferably made from polymeric materials as discussed previously.

As shown in FIG. 1a, the supporting container 12 comprises of a first side wall 14 connected to a portion of a second side wall 15 along a peripheral seal to define a containment area 24, each side wall having an interior surface 16 and 18, respectively.

At least one of the side walls is constructed from a flexible, gas permeable material. The other side wall can be constructed from the same flexible, gas permeable material, or it can be constructed from a different flexible, gas permeable material. Alternately, the second side wall can be constructed from a non-gas permeable, but flexible material. In the embodiment of FIG. 1a, the material of the side walls is a monolayer structure. In other preferred embodiments, the material of one or both side walls can be multilayer structures.

The supporting container 12 preferably has a flexural modulus from about 5,000 to about 300,000 psi as measured according to ASTM D-790. More preferably, the flexural modulus of the container is within the range of 10,000-200,000 psi, and most preferably, 10,000-30,000 psi.

The supporting container 12 preferably has the following permeability characteristics: (1) an oxygen permeability within the range of about 7 to about 30 Barrers, and more preferably 9 to 15 Barrers; (2) a carbon dioxide permeability within the range of 40 to 80 Barrers; (3) a nitrogen permeability of 10 to 100 Barrers, and (4) a water vapor transmission rate of not more than 20 (g mil/100 in²/day). The gas permeability of the supporting container 12 can be adjusted to best match the requirements of the cells being cultured in the container by varying the material of the container, the thickness of the container, or the thickness of each of the layers if a multilayer structure is used.

It is preferred that at least a portion of the supporting container 12 is optically transparent, with an optical clarity preferably within the range of about 0.1% to about 10% as measured by a Hazometer in accordance with ASTM D1003, to allow observation of the cells during the culture. It is more preferred that a substantial portion of the supporting container 12 is optically transparent. In a preferred embodiment, one of the side walls 14 or 15 is optically transparent. In another preferred embodiment, both side walls 14 and 15 are optically transparent. The supporting container 12 should also be able to withstand radiation sterilization at radiation levels commonly used in the industry for sterilization.

The method for fabricating the flexible, gas permeable supporting container is disclosed in the commonly assigned U.S. Pats. No. 4,939,151 and No. 5,935,847. The supporting container 12 includes a body that is constructed from a first side wall 14 and a second side wall 15. The side walls 14 and 15 are sealed along their edges to define a containment area 24 for containing the cell culture media and cells. The side walls 14 and 15 may be sealed by any conventional means such as using heated die and platen which may be followed by a chill die and platen as is well known in the industry. Also, the side walls 14 and 15 may be sealed using inductive welding which also is known in the industry. For containers constructed from films having the polymer alloy including the dimer fatty acid polyamide, radio frequency techniques may be used. However, the present invention should not be construed to be limited to using any one of these fabrication techniques unless otherwise specified in the claims.

Supporting containers 12 fabricated using these preferred methods have been found to be sufficiently strong to withstand centrifuging even over an extended period of time at high gravitational forces.

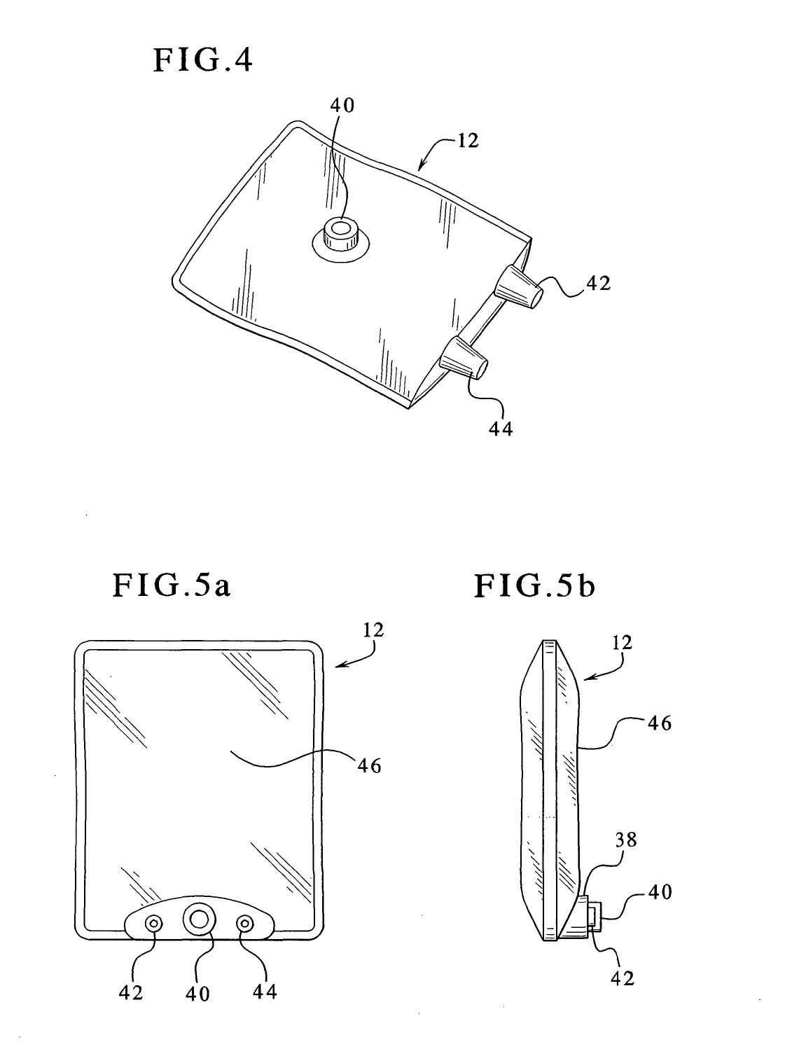

In an embodiment shown in FIG. 4, the supporting container 12 preferably has a fill port 40 and two access ports 42 and 44. Although two access ports are illustrated in the embodiment of FIG. 4, more or less access ports can be utilized. The fill port is for introducing the fibrin matrix into the supporting container 12, and for facilitating the introduction of gas(es) and/or media growth factors, and cells. The fill port 40 may be constructed to accommodate a fibrin delivery device. The access ports are for the removal of the cells/tissue at an appropriate time. Of course, any number of access ports can be provided as well as a tube set assembly, or the like. Preferably, the access ports are constructed from a material that can be easily sealed. Accordingly, after access to the container, the access ports can be sealed, enclosing the cell culture within the container 10. In a preferred embodiment, the fill port 40 and the access ports 42, 44 are constructed from a material that can be sonically welded. Preferably, the fill port 40 and the access ports 42, 44 are constructed from a polyolefin. In an embodiment, the fill port 40 and access port 42, 44 are constructed from a high density polyethylene.

In one embodiment, the supporting container 12 includes a fitment 38 as illustrated in FIG. 5a, which is disclosed in the commonly assigned U.S. Pat. No. 4,910,147, which is hereby incorporated herein by reference and made a part hereof. The fitment 38 provides means for accessing a containment area defined by the container for filling the container and/or accessing the contents of a filled container. To this end, in the embodiment illustrated in FIG. 5a, the fitment 38 includes a fill port 40 and access ports 42, 44. It should be noted that although two access ports are illustrated on the fitment 38, more or less access ports can be utilized. Furthermore, if desired, the fill port 40 and access ports 42, 44 can be secured to separate fitments. In constructing the supporting container 12, in an embodiment, holes are punched in the film and the fill port 40 and access ports 42, 44 are inserted therethrough and a top portion of the body of the fitment 38 is sealed to the film.

The fill port 40 is utilized to fill the container 10 with the fibrin matrix, cell culture media and the cells. Preferably, the fill port 40 is constructed from a material that can be easily sealed. Accordingly, after the container 10 has been filled with cell culture media, the fill port 40 can be sealed enclosing the cell culture media within the container 10. In a preferred embodiment, the fill port 40 is constructed from a material that can be sonically welded. Preferably, the fill port 40 is constructed from a polyolefin. In an embodiment, the fill port 40 is constructed from a high density polyethylene.

Typically, in use, the supporting container 12 is filled by having a nozzle or other means inserted in the fill port 40 and cell culture media fed therein. The nozzle or other means is then removed from the fill port and the fill port is sonically welded.

In a preferred embodiment, the fill port 40 is adapted for receiving a delivery device to load the fibrin into the supporting container 12.

The access ports 42, 44 provide a means for accessing the contents of the container 10. To this end, the access ports 42, 44 are designed to receive a standard spike/luer. Preferably, the access ports 42, 44 are sealed by a removable cap and include a pierceable membrane that is pierced by a spike, or like means, when the container is accessed. Of course, other means of accessing the container via the access ports 42, 44 can be utilized.

As illustrated in FIG. 5b, in contrast to a standard fitment and port arrangement, the supporting container 12 of this embodiment is constructed so that the fitment 38, and specifically the ports 40, 42, 44 extend outwardly from a face 46 of the supporting container 12. In typical flexible containers, the fitment or ports extend from the bottom edge of the container in a plane that is substantially parallel to a plane defined by the face 46 of the container. By extending the ports 40, 42, and 44 of the fitment 38 outwardly from the face 46 of the supporting container 12, i.e., normal to a plane defined by the face 46 of the supporting container 12, the container can be easily and cost effectively fabricated and filled with cell culture media utilizing a semi-automatic, aseptic fill machine. Further, the fitment arrangement 38 provides a supporting container 12 from which the cell culture media stored therein can be easily accessed.

The access to the containers 10, 12 (FIGS. 1b, 4 and 5) is not limited to the access ports or the fitment described above. Other methods may also be suitable for accessing the containers 10, 12. For example, the fitment 38 illustrated in FIGS. 5a and 5b can be replaced with an end port commonly used in intravenous (IV) containers.

III. Fibrin Matrix Modified Container

While the supporting container 12 provides the physical properties of flexibility, gas permeability, and optical clarity, the fibrin matrix provides the requirement of biocompatibility between the container and the cultured cells.

As shown in FIG. 1b, the fibrin matrix 20 covers a least a portion of the interior surface of one of the side walls of the supporting container. Preferably, the fibrin matrix 20 covers a substantial portion of the interior surface 16 or 18, and even more preferably substantially the entire interior surface, and most preferably substantially the entire surfaces of both sidewalls. In one embodiment, the fibrin matrix covers a substantial portion of the interior surfaces 16 and 18 of both side walls 14 and 15.

The fibrin matrix 20 is composed of polymerized fibrin monomers. Fibrin is naturally found in blood clots to prevent further bleeding from an injured site. It is also commercially available as a sealant or glue for wound healing and for hemostasis. An example of a commercial fibrin product is available from Baxter Healthcare Corporation (Deerfield, Ill.) under the trade name TISSEEL™. The fibrin matrix 20 of the present invention provides an environment conducive for cell growth, particularly the growth of “anchorage dependent” cells. Pores, present in the matrix between the fibrin polymers, provide a location for the cells to attach. The fibrin matrix 20, therefore, forms a biocompatible cell growth surface. Accordingly, the film and the synthetic polymer side walls do not have to provide this function.

The physical characteristics of the fibrin matrix 20 (e.g., pore size, density, thickness, etc.) can be varied to meet the specific requirements of the cells to be cultured.

The chemistry of the formation of the natural fibrin matrix in blood clots is well discussed in detail in references such as Bach et al., “Fibrin Glue As Matrix For Cultured Autologous Urothelial Cells In Urethral Reconstruction”, Tissue Engineering Vol. 7 No. 1, p. 45-53, 2001. In summary, fibrinogen is proteolytically cleaved to form fibrin monomers by thrombin. Factor XIII is also proteolytically cleaved by thrombin to form the activated factor XIII, factor XIIIa, which catalyzes the polymerization of the fibrin monomers to form the fibrin clot.

There are many approaches to preparing the fibrin matrix 20 in the present invention. The methods to prepare fibrin matrices are well known to those skilled in the art. The matrix is generally formed by the polymerization of fibrin monomers catalyzed by factor XIIIa. In one of the embodiments, the fibrin matrix is prepared by mixing a first solution containing fibrinogen and factor XIII with a second solution containing thrombin and calcium. The thrombin, in the presence of calcium, proteolytically cleaves the fibrinogen to form fibrin monomers, and the factor XIII to form factor XIIIa. The factor XIIIa formed catalyzes the polymerization of the fibrin monomers to form the fibrin matrix. The characteristics of the fibrin matrix can be varied by varying the concentrations of the various components in the first and the second solutions and the temperature of the reaction.

In a preferred embodiment, the concentration of the fibrinogen in the first solution is from about 2.0 to about 20 mg/mL, the concentration of the factor XIII in the first solution is from about 10 to about 40 IU/mL, the concentration of the thrombin in the second solution is from about 2.5 IU/mL to about 50 IU/mL, and the concentration of the calcium in the second solution is from about 40 to about 100 mmoles/mL. Approximately 0.5-1.0 mLs of the first solution is mixed with 0.5-1.0 mLs of the second solution to form a fibrin-forming mixture. The polymerization reaction takes place at room temperature in 1-5 minutes and is complete in about 5-15 minutes at 37° C. The fibrin matrix formed in this embodiment has a pore size of about 0.5-5.0 μm in diameter. Of course, those of skill in the art will recognize that a variety of other constituents may be included in the first or second solution, and the concentrations of the various components in the first or second solution may be substituted or may vary in concentrations according to the desired physical property of the fibrin matrix.

The first solution of fibrinogen and factor XIII and the second solution of thrombin and calcium can be mixed before applying to the inner surface of the supporting container 12, or the solutions can be applied separately onto the inner surface of the container without mixing. The polymerization of fibrinogen monomers takes place when the solutions are in contact on the inner surface of the container.

The fibrinogen is generally derived from mammalian plasma, preferably human plasma. The fibrinogen can also be prepared by any of the recombinant methods known. The factor XIII is generally derived from mammalian plasma, preferably human plasma. The factor XIII can also be a recombinant factor XIII made from any known methods. The thrombin is generally from plasma of mammalian origin, preferably from bovine plasma, and more preferably from human plasma. The thrombin can also be a recombinant thrombin prepared by any known methods.

IV. Fibrin Delivery Device

In a preferred embodiment, the fibrin is loaded into the supporting container 12 by a delivery device via the fill port 40 which may be customized in size, shape, and geometry to accommodate the delivery device. Numerous fibrin delivery devices are commercially available. These devices mix the fibrinogen first solution and the thrombin second solution to form the fibrin-forming mixture and then apply the mixture onto the inner surface of the container to form the fibrin matrix. It is contemplated that these devices would work or could be made to work to load the fibrin into the supporting container 12. It is also possible to utilize a delivery device that can spray or to otherwise deposit the thrombin solution and the fibrinogen solution, either sequentially or simultaneously, for forming the fibrin in situ on the container surface. This helps reduce the occurrences of clog-forming occlusions of fibrin material that can occur when the fibrinogen solution and the thrombin solution are mixed in the device.

One such device is disclosed in U.S. Pat. No. 4,978,336, which discloses a dual syringe system. A device made by the assignee of the '336 patent, Hemaedics, Inc., is sold under the tradename DUOFLO™. Each syringe distal end is attached to a common manifold having a mixing chamber. Fibrinogen and thrombin solutions are mixed in the manifold prior to application to a wound or other surface. The manifold has a discharge tip for delivering the mixed solution onto a surface.

The commonly assigned U.S. Pat. No. 4,631,055 discloses another thrombin and fibrinogen delivery device having two syringes mounted in a holding frame in parallel spaced relationship. A conical portion of a distal end of each syringe is inserted into a connecting head. In one embodiment of the '055 patent, mixing of fluids contained in each syringe occurs inside the connecting head and in another embodiment the mixing of the fluids occurs outside the mixing head. The connecting head also includes a channel to supply medicinal gas under pressure. The medicinal gas contacts the fluids at a mouth of the connecting head and conveys the fluids contained in the syringes to a surface.

A preferred delivery device for introducing the fibrin matrix into the supporting container 12 is a spraying device such as the one disclosed in the commonly assigned U.S. Pat. No. 5,989,215, which is hereby incorporated by reference and made a part hereof.

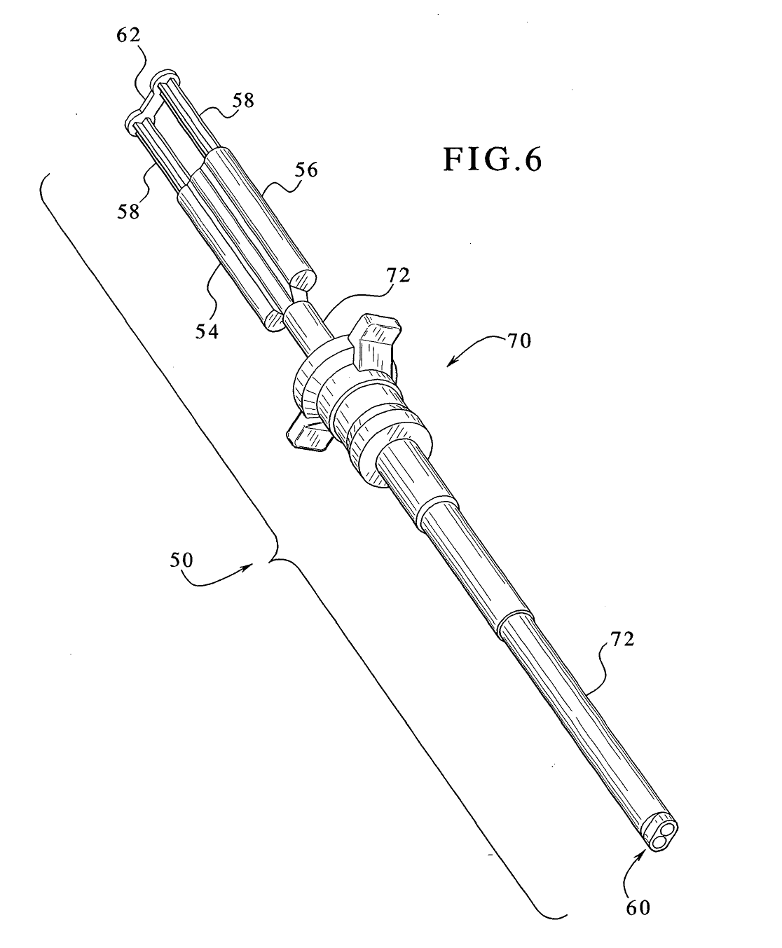

FIG. 6 is a perspective view of an embodiment of the delivery device adapted for use in delivering fibrin through the fill port 40. This device is particularly adapted for inserting into a surgical opening of an animal body to provide access to a cavity of the animal.

As illustrated in FIG. 6, the delivery device 50 has tubings which extend from the first and second containers 54 and 56 through a sleeve 72. The first container 54 has a first opening, and the first container 54 is adapted to contain the first biochemically reactive fluid. The second container 56 has a second fluid opening adjacent the first fluid opening; the second container 56 is adapted to contain the second biochemically reactive fluid. The containers 54 and 56 are preferably syringes and are attached together or are integral with one another to define a single unit. It is also preferable that the containers 54 and 56 have equal volumes. The spray unit 60 is in fluid communication with the first container 54 and the second container 56, the spray unit 60 being capable of separately atomizing the first and second biochemically reactive fluids into an aerosol with at least one energy source of a liquid energy, a mechanical energy, a vibration energy, and an electric energy. A fluid pressurizer is associated with the first and second containers for pressurizing the first and the second biochemically reactive fluids for delivery under pressure through the spray unit onto a surface. Wherein the first and second biochemically reactive fluids first mix on the surface. This device does not use pressurized gas. The pressurizer in this embodiment is a dual plunger having two horizontally spaced plungers 58 mechanically coupled at one end by a crossbar 62. The sleeve 72 extends through a trochar 70 which is inserted into the fill port 40. In this fashion the spray unit 60 may be directed into the containment area 24 of the supporting container 12. The tip of the spray unit 60 may be customized for docking the delivery device 50 to the fill port 40, and likewise, the fill port 40 may be customized to receiving and securing the delivery device 50 during the delivery of the fibrin solution.

V. Method for Loading Fibrin into the Supporting Container

Various methods can be used for loading fibrin into the supporting container 12. In one embodiment of the invention, the fibrin is loaded into the supporting container with the delivery device 50 disclosed in the '215 patent via an access port. The method is as follows.

The first step involves docking of the delivery device 50 to the fill port 40 of the supporting container 12. As discussed earlier, the tip of the spray unit 60 of the delivery device 50 can be adapted for docking to the fill port 40 of the supporting container 12. For illustration in this example, the fibrin solution is delivered into the supporting container via a fill port 40 on the container. FIG. 4 is a perspective view of a container with a single fill port 40 for receiving the delivery device 50. The tip of the spraying unit 60 should be within the containment area 24 of the supporting container 12.

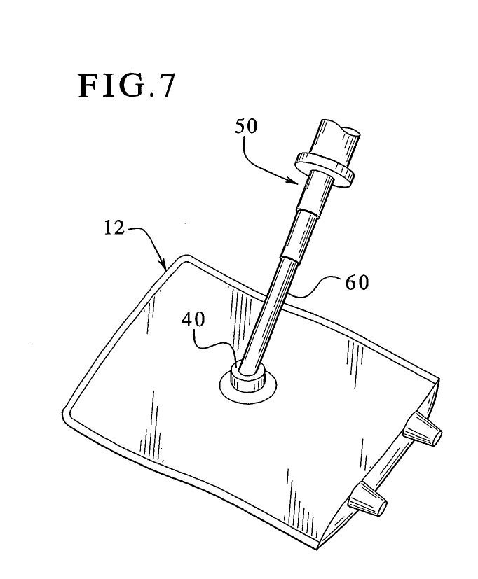

FIG. 7 is a schematic drawing showing a partial view of the delivery device 50 docking to the fill port 40 of the supporting container 12 which is ready to spray the fibrin solution onto the inner surface of the side wall of the supporting container 12.

Before loading the fibrin into the supporting container 12, it is preferred that the container 12 be inflated with a gas, such as air, nitrogen, hydrogen, carbon dioxide, helium, and the like. The delivery device 50 preferably can also deliver the gas to inflate the flexible supporting container 12. In one embodiment, the gas is delivered into the supporting container via a gas line extended through the trocar 70.

A first fibrinogen-containing solution is then loaded into the first container 54 of the delivery device 50, followed by a second thrombin-containing solution loaded into the second container 56 of the delivery device 50. Preferably, equal volumes of the first solution and the second solution be loaded into their respective containers. In case the different volumes of the first and the second solution should be simultaneously mixed, it will be known in the art which measures have to be taken in order to ensure that a homogenous mixture is obtained. The solutions in the containers 54 and 56 are pressurized to deliver streams of the solutions under pressure through the spray unit 60. The resulting mixture is spread over onto the inner surface of the side wall of the supporting container which is tilted to cover the entire surface as far as possible before the formation of the three-dimensional fibrin network starts. The mixture is then incubated at appropriate conditions to allow the mixture to set completely to form a fibrin matrix with desirable physical characteristics. Preferably, completion of the conversion of fibrinogen to fibrin is achieved by incubation of the fibrin matrix at the physiological temperature, i.e., 37° C., for 200 minutes.

For preparing a fibrin matrix with a higher concentration of thrombin, it may not be desirable to mix the first fibrinogen-containing solution with the second thrombin-containing solution at the same time since the clotting time is much reduced at higher thrombin concentrations. In this case, the fibrin matrix can be prepared by first applying the first solution onto the inner surface of the supporting container followed by applying the second solution separately. The two solutions are in contact on the inner surface of the side wall of the supporting container 12. In order to obtain a fibrin matrix having a regular thickness and a homogenous structure the first, aqueous, fibrinogen-containing solution should be uniformly distributed over the entire inner surface.

It is, of course, recognized that the preliminary process steps of the two processes described above are preferred laboratory procedures that might be readily replaced with other procedures of equivalent effect.

In one embodiment of the present invention, the fibrin matrix can also be introduced into the supporting container 12 as a preformed, dry fibrin fleece. In one embodiment, the fibrin fleece can be introduced into the supporting container 12 through the fill port 40. In another embodiment, the fibrin fleece can be placed between the two side walls 14 and 15 of the supporting container 12 before the side walls 14 and 15 are sealed. The dry fibrin fleece in the supporting container 12 can be rehydrated to form the fibrin matrix 20 within the supporting container 12.

The method of making the fibrin fleece is disclosed in the co-pending and commonly assigned U.S. Patent Application No. 20020131933 A1, which is incorporated herein by reference and made a part hereof. The steps of the process for preparing the fibrin fleece are: (1) providing a solution containing fibrin or fibrinogen materials; (2) polymerizing the fibrin or fibrinogen, preferably a polymerization with at least partial cross-linking of the fibrin or fibrinogen materials in the presence of a calcium blocking or inhibiting agent (preferably an anticoagulant); and (3) lyophilizing the polymerized fibrin or fibrinogen. The resulting fibrin or fibrinogen material is in its substantially dry form.

In yet another embodiment of the present invention, a uniform and homogenous fibrin layer is formed on the inner surface of the side wall of the supporting container 12. Generally, when fibrin layers are formed by simple immersion, a compact fibrin layer is formed which has little of no fibrin in the support pores. Alternatively, the fibrin is only found in the pores having great diameters and there is substantially no fibrin found in the pores of small diameter. This lack of uniformity is known to affect cell attachment. The homogenous layer of fibrin formed is characterized by the lack of fibrinogen unbound from the fibrin layer. This uniform fibrin layer facilitates the attachment of cells. The method of making the uniform homogenous fibrin layer is disclosed in co-pending and commonly assigned U.S. patent application Ser. No. 09/831,121, which is incorporated herein by reference and made a part hereof.

VI. Method for Culturing Cells in the Fibrin Matrix Enhanced Cell Culture Container

The cell culture container of the present invention is most applicable in culturing of “anchorage dependent” cells, which is also known as “adherent” cells. However, the cell culture container 10 can also be used to culture “non-adherent” cells. It is known that certain “non-adhering” cells grow better in an adhering surface such as the one offered by the fibrin matrix in the present invention. The container is particularly applicable to culturing mammalian cells for expansion and transplantation in a closed system. By employing a closed system, the invention is well suited for applications involving therapeutic transplantation of cultured cells. The container 10 also presents the potential of a system for generating formed tissue, not just individual cells and small aggregates.

An example of the cells that can benefit from the closed system flexible, gas permeable container of the present invention is the human pancreatic islets of Langerhans (islets). Another example is the insulin-producing endocrine cells. Both the islet cells and the insulin-producing cells are used in preparation for transplantation. Pancreatic islet cells are currently grown by conventional open-system methods. An example of progenitor cells that can be cultured in the container of the present invention is the pancreatic duct- or islet-derived progenitor cells. Such cells have been shown to successfully expand and differentiate into insulin-producing endocrine cells, a potential source for transplantation.

Other cells that are contemplated for use with the present invention include, but are not limited to, oral mucosa cells, peripheral nerve cells, muscle cells, trachea cells, cartilage cells, meniscal cells, corneal cells, fat cells, cardiovascular cells, urothelial cells, skin cells, and bone cells.

The cell culture container 10 must be sterilized before use. In a preferred form of the invention, the cell culture container 10 or the supporting container 12 is radiation sterilized. Other sterilization method, such as steam autoclaving, can also be employed. In one embodiment, the supporting container 12 is sterilized. The fibrin material is pre-sterilized and is introduced through the fill port 40 to form the matrix on at least one side wall of the supporting container 12. The fibrin matrix may be introduced into the sterile supporting container 12 using one of the many methods disclosed herein or known in the art, such as using a delivery device disclosed in U.S. Pat. No. 5,989,215, or as a fibrin fleece as disclosed in the U.S. Patent Application No. 20020131933 A1. A cell line in an appropriate cell culture medium is then introduced into the container, preferably via the fill port 40. The formation of the fibrin matrix within the container and the introduction of the cell line into the container are conducted under aseptic conditions. The fill port 40 may then be sealed if desired. However, the fill port 40 can include an injection site or port tube and therefore is not sealed. Alternately, the container 10 can be radiation sterilized after the formation of the fibrin matrix in the supporting container 12. The cells are then allowed to grow in the container under appropriate conditions such as 37° C. under an atmosphere of a mixture of oxygen and 5-10% carbon dioxide. Cell culture medium containing a source of either human or animal serum, preferably at 5-20% final serum concentration. The cell culture medium preferably contains growth factors to facilitate the culture and/or adhering of the cells to the matrix. Suitable cell growth factors include Epidermal Growth Factor (EGF), Keratinocyte Growth Factor (KGF), or Hepatocyte Growth Factor (HGF). In a preferred form of the invention, the growth factor is serum proteins. A preferred source of serum proteins is fetal calf serum or human serum.

VI. Method of Preparing Cells or Tissue for Cell Culture

The method for preparing cells or tissue for cell culture varies from the cells or tissue used. The methods are generally known by those of skill in the art of cell culture. For example, pancreatic caveric tissue is obtained surgically from donors. The tissue is prepared in a digestion mixture primarily consisting of collagenase enzyme. After 1-2 hours of perfusion the tissue breaks down into smaller size tissue samples. These smaller size tissue samples are harvested by centrifigation on a ficoll gradient. The less buoyant particles are sedimented while the more buoyant particles are harvested and used for transplant to a recipient. The sedimented portion “leftover” fraction is then suspended in cell culture medium or a suitable storage solution for transport. This fraction is then set aside until the cell culture container or supporting container is prepared.

### EXAMPLES

### Example 1

### A Flexible, Gas Permeable Cell Culture Container with Fibrin Matrix Using a PL269 Cryocyte™ Bag as the Supporting Container

Cryocyte™ bag is supplied by Baxter International Inc. (Baxter Code No. R4R9951, PL269). A fibrin matrix is formed with TISSEEL™ components on the bag surface being combined at a final concentration of 10 mg/mL Sealer Protein Concentrate and 50 IU/mL thrombin, respectively. The fibrin matrix is prepared and the concentration of the fibrinogen in the first solution is from about 2.0 to about 20 mg/mL, the concentration of the thrombin in the second solution is from about 2.5 IU/mL to about 50 IU/mL, and the concentration of the calcium in the second solution is from about 40 to about 100 mmoles/mL. Approximately 0.5-1.0 mLs of the first solution is mixed with 0.5-1.0 mLs of the second solution to form a fibrin-forming mixture. The polymerization reaction takes place at room temperature in 1-5 minutes and is complete in about 5-15 minutes at 37° C. The fibrin matrix formed in this embodiment has a pore size of about 0.5-5.0 μm in diameter.

### Example 2

### Pancreatic Cell Culture in a Flexible, Gas Permeable Cell Culture Container with Fibrin Matrix Using PL269 Cryocyte™ Bags as Supporting Containers.

The fibrin treated PL269 Cryocyte™ bag is seeded with cultured pancreatic cells. The formation of a fibrin matrix in the bag is confirmed with scanning electron microscopy (SEM). As shown in FIG. 5, the SEM photo shows the fibrin matrix with cells adhering to the matrix.

### Example 3

### Cell Culture of Pancreatic Cells in PL269 Bags with and without Fibrin Matrix, and in T-75 Non-Gas Permeable Rigid Polystyrene Flasks.

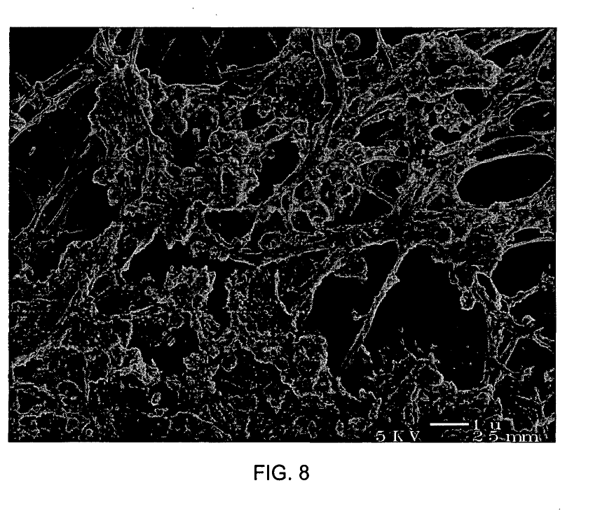

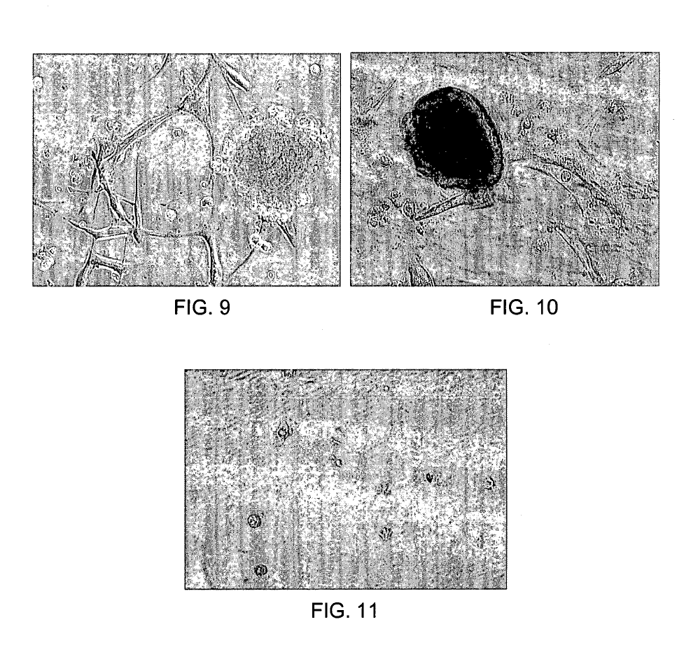

Pancreatic cells are cultured in PL269 bags with and without fibrin matrix, and in T-75 polystyrene flasks for 4 days. The cells in the bags are observed with a phase contrast microscope. The cells appear to be fibroblasts. The adherence of the cells within the fibrin matrix in the PL269 bag (FIG. 7) is comparable to the T-75 flask (FIG. 6). The PL269 bag without the fibrin matrix has no apparent attached cells (FIG. 8). Floating cells can be seen throughout the culture medium, not adhering to any of the bag surfaces.

The flask provides a positive control, confirming the presence and appearance of “anchorage dependent” cells that are maintained in an “open” method of culture. The fibrin matrix treated PL269 bag shows cells having a comparable physical appearance, which are maintained under a closed system culturing process.

### Example 4

### Adhesion of Islet Cells to Fibrin Matrix in the Presence of Serum Proteins

Pancreatic islet cells are cultured in PL269 bags with or without fibrin matrix and in T-75 polystyrene flasks in the presence or absence of serum proteins. The cells display varying levels of adhesion based upon the combination of fibrin matrix and the presence of serum proteins.

| Container | Serum Proteins | Fibrin Matrix | Cell Adherence | |
|---|---|---|---|---|
| After 4 days of | | | | |
| culture | | | | |
| T-Flask | No | No | None | 0% |
| PL269 bag | No | Yes | Fair | 20-25% |
| PL269 bag | Yes | No | Poor | 1-5% |
| PL269 bag | Yes | Yes | Excellent | 80-90% |
| After 28 days of | | | | |
| culture | | | | |
| T-Flask | Yes | Yes | Excellent | 85-90% |
| PL269 bag | Yes | Yes | Good | 60-70% |
| PL269 bag | Yes | No | None | 0% |

This phenomenon of cell adherence is a function of serum proteins and fibrin matrix has been shown to be independent of bag material, as long as materials have comparable gas permeability and biocompatibility.

### Example 5

### Flexible, Gas Permeable Multilayer Cell Culture Container with Fibrin Matrix

It is contemplated that the flexible, gas permeable cell culture supporting container be constructed from a multilayer structure in the product designation PL2417® available from Baxter Healthcare Corporation (Deerfield, Ill.). The structure comprises an ultra-thin polystyrene layer and a second polymeric layer. A fibrin matrix can be incorporated into a supporting container constructed with this multilayer structure to form a gas permeable, flexible cell culture container.

It is understood that, given the above description of the embodiments of the invention, various modifications may be made by one skilled in the art. Such modifications are intended to be encompassed by the claims below.

1. A cell culture container comprising:
a supporting container comprising a first side wall connected to a portion of an opposing second side wall along a peripheral seal to define a containment area, each side wall having an interior surface, the first side wall being constructed from a gas permeable material selected from the group consisting of: polymeric material, paper, and fabric, the first side wall having a gas permeability sufficient to permit cellular respiration, and the second side wall being constructed from a material selected from the group consisting of: polymeric material, paper, fabric, and foil; and
a fibrin matrix layer on a portion of the interior surface of the first side wall or the second side wall of the supporting container.

2. The cell culture container of claim 1 wherein the gas permeable material is selected from the group consisting of: ethylene vinyl acetate copolymers, polyolefins, polyamides, polyesters, styrene and hydrocarbon copolymers, and fluorocarbon elastomers.

3. The cell culture container of claim 1 wherein the polymeric material of the first side wall or the second side wall of the supporting container is a multiple-component polymer blend.

4. The cell culture container of claim 3 wherein at least one of the components of the multiple-component polymer blend is a styrene and hydrocarbon copolymer.

5. The cell culture container of claim 1 wherein the gas permeable material is a monolayer structure.

6. The cell culture container of claim 1 wherein the gas permeable material is a multilayer structure.

7. The cell culture container of claim 6 wherein the multilayer structure comprises:
a first layer comprising a first ethylene vinyl acetate copolymer, the first layer having a first surface and a second surface; and
a second layer adhering to the first surface of the first layer, the second layer comprising a second ethylene vinyl acetate copolymer;
wherein the second surface of the first layer forms the interior surface of the supporting container.

8. The cell culture container of claim 7 wherein the first ethylene vinyl acetate copolymer having a vinyl acetate content of greater than 18% by weight of the copolymer.

9. The cell culture container of claim 7 wherein the second ethylene vinyl acetate copolymer having a vinyl acetate content of less than 18% by weight of the copolymer.

10. The cell culture container of claim 7 wherein the first ethylene vinyl acetate copolymer having a vinyl acetate content of about 18% by weight of the copolymer.

11. The cell culture container of claim 7 wherein the second ethylene vinyl acetate copolymer having a vinyl acetate content of about 9% by weight of the copolymer.

12. The cell culture container of claim 6 wherein the multilayer structure comprises:
a first layer comprising polystyrene having a thickness within the range of 0.0001 inches to about 0.0010 inches; and
a second layer adhering to the first layer, the second layer comprising a polymeric material selected from the group consisting of ethylene vinyl acetate copolymers, polyolefins, polyamides, polyesters, styrene and hydrocarbon copolymers, fluorocarbon elastomers, the second layer having a thickness within the range of 0.004 inches to about 0.025 inches.

13. The cell culture container of claim 12, wherein the polymeric material of the second layer is a multi-component polymer blend.

14. The cell culture container of claim 13, wherein at least one of the components of the multi-component polymer blend is a styrene and hydrocarbon copolymer.

15. The cell culture container of claim 12, wherein the fibrin matrix is positioned on a portion of the polystyrene layer covering substantially an entire surface of the polystyrene layer.

16. The cell culture container of claim 1, wherein the second side wall is constructed from a gas permeable material selected from the group consisting of: polymeric materials, paper, and fabric.

17. The cell culture container of claim 16, wherein the polymeric material of the second side wall is selected from the group consisting of: ethylene vinyl acetate copolymers, polyolefins, polyamides, polyesters, styrene and hydrocarbon copolymers, and fluorocarbon elastomers.

18. The cell culture container of claim 17, wherein at least one of the components of the multi-component polymer blend is a styrene and hydrocarbon copolymer.

19. The cell culture container of claim 16, wherein the gas permeable material is a monolayer structure.

20. The cell culture container of claim 16, wherein the gas permeable material is a multilayer structure.

21. The cell culture container of claim 20, wherein the multilayer structure comprises:
a first layer comprising a third ethylene vinyl acetate copolymer with a vinyl acetate content of greater than 18% by weight of the copolymer, the first layer having a first surface and a second surface; and
a second layer adhering to the first surface of the first layer, the second layer comprising a fourth ethylene vinyl acetate copolymer with a vinyl acetate content of from less than 18% by weight of the copolymer,
wherein the second surface of the first layer forms the inner surface of the container.

22. The cell culture container of claim 21, wherein the a vinyl acetate content of the fourth vinyl acetate copolymer in the first layer is about 18% by weight of the copolymer.

23. The cell culture container of claim 21 wherein the a vinyl acetate content of the fifth vinyl acetate copolymer in the second layer is about 9% by weight of the copolymer.

24. The cell culture container of claim 20, wherein the multilayer structure comprises:
a first layer comprising polystyrene having a thickness within the range of 0.0001 inches to about 0.0010 inches; and
a second layer adhering to the first layer, the second layer comprising a polymeric material selected from the group consisting of ethylene vinyl acetate copolymers, polyolefins, polyamides, polyesters, styrene and hydrocarbon copolymers, fluorocarbon elastomers, the second layer having a thickness within the range of 0.004 inches to about 0.025 inches.

25. The cell culture container of claim 24, wherein the polymeric material of the second layer is a multi-component polymer blend.

26. The cell culture container of claim 25, wherein at least one of the components of the multi-component polymer blend is a styrene and hydrocarbon copolymer.

27. The cell culture container of claim 1, wherein the container having an oxygen permeability of from about 9 to about 15 Barrers, a carbon dioxide permeability of from about 40 to about 80 Barrers, a nitrogen permeability of from about 10 to about 100 Barrers, and a water vapor transmission rate of less than about 20 (g mil/100 in²/day).

28. The cell culture container of claim 1, wherein the first side wall and the second side wall having a flexural modulus of from about 10,000 to about 30,000 psi as measured according to ASTM D-790.

29. The cell culture container of claim 1, wherein at least a portion of the container is optically clear.

30. The cell culture container of claim 1, wherein a substantial portion of the container is optically clear.

31. The cell culture container of claim 1, wherein the container is radiation sterilizable.

32. The cell culture container of claim 1, wherein the container further comprising at least one port providing access to the containment area.

33. The cell culture container of claim 1, wherein the fibrin matrix extends over substantially an entire surface of the interior surface of at least one of the side walls.

34. The cell culture container of claim 1, wherein the fibrin matrix is on at least a portion of the interior surface of each of the side walls.

35. The cell culture container of claim 1, wherein the fibrin matrix is three-dimensional with pore sizes of from about 0.5 to about 5.0 um in diameter.

36. The cell culture container of claim 1, wherein the fibrin matrix is formed by cross-linking fibrin or fibrinogen.

37. The cell culture container of claim 1, wherein the fibrin matrix is prepared by mixing a first solution comprising fibrinogen and factor XIII with a second solution comprising thrombin and calcium to form the fibrin matrix.

38. The cell culture container of claim 37, wherein the fibrinogen is derived from mammalian plasma.

39. The cell culture container of claim 38, wherein the mammalian plasma is human plasma.

40. The cell culture container of claim 37, wherein the fibrinogen is a recombinant fibrinogen.

41. The cell culture container of claim 37, wherein the factor XIII is derived from mammalian plasma.

42. The cell culture container of claim 41, wherein the mammalian plasma is human plasma.

43. The cell culture container of claim 37, wherein the factor XIII is a recombinant factor XIII.

44. The cell culture container of claim 37, wherein the thrombin is derived from mammalian plasma.

45. The cell culture container of claim 44, wherein the mammalian plasma is selected from the group consisting of bovine plasma and human plasma.

46. The cell culture container of claim 37, wherein the thrombin is a recombinant thrombin.

47. The cell culture container of claim 37 wherein the concentration of fibrinogen in the first solution is from about 2.0 to about 20 mg/mL, the concentration of the factor XIII in the first solution is from about 10 to about 40 IU/mL, the concentration of the thrombin in the second solution is from about 2.5 IU/mL to about 50 IU/mL, and the concentration of the calcium in the second solution is from about 40 to about 100 mmoles/mL. Approximately 0.5-1.0 mLs of the first solution is mixed with 0.5-1.0 mLs of the second solution to form a fibrin-forming mixture. The polymerization reaction takes place at room temperature in 1-5 minutes and is complete in about 5-15 minutes at 37° C. The fibrin matrix formed in this embodiment has a pore size of about 0.5-5.0 μm in diameter.

48. A cell culture container comprising:
a supporting container comprising a first side wall connected to a portion of an opposing second side wall along a peripheral seal to define a containment area, each side wall having an interior surface, the first side wall and the second side wall are constructed from an ethylene vinyl acetate copolymer having a gas permeability sufficient to permit cellular respiration; and
a fibrin matrix layer on a portion of the interior surface of the first side wall or the second side wall of the supporting container.

49. The cell culture container of claim 47, wherein the ethylene vinyl acetate copolymer is a multilayer structure comprising:
a first layer comprising a fifth ethylene vinyl acetate copolymer with a vinyl acetate content of greater than 18% by weight of the copolymer, the first layer having a first surface and a second surface; and
a second layer adhering to the first surface of the first layer, the second layer comprising a sixth ethylene vinyl acetate copolymer with a vinyl acetate content of less than 18% by weight of the copolymer;
wherein the second surface of the first layer forms the interior surface of the supporting container.

50. The cell culture container of claim 48, wherein the vinyl acetate content of the sixth ethylene vinyl acetate copolymer is about 18% by weight of the copolymer, and the vinyl acetate content of the seventh ethylene vinyl acetate copolymer is about 9% by weight of the copolymer.

51. A cell culture container comprising:
a supporting container comprising a first side wall connected to a portion of an opposing second side wall along a peripheral seal to define a containment area, each side wall having an interior surface, the side walls are constructed from a multilayer gas permeable polymeric structure having a gas permeability sufficient to permit cellular respiration, and the multilayer polymeric structure comprising:
a first layer comprising polystyrene having a thickness within the range of 0.0001 inches to about 0.0010 inches; and
a second layer adhering to the first layer, the second layer comprising a polymeric material selected from the group consisting of ethylene vinyl acetate copolymers, polyolefins, polyamides, polystyrene and hydrocarbon copolymers, the second layer having a thickness within the range of 0.004 inches to about 0.025 inches; and
a fibrin matrix layer on a portion of the interior surface of the first side wall or the second side wall of the supporting container.

52. The cell culture container of claim 51, wherein the polymeric material of the second layer is a multi-component polymer blend.

53. The cell culture container of claim 52, wherein at least one of the components of the multi-component polymer blend is a styrene and hydrocarbon copolymer.

54. A method of culturing cells, the method comprising the steps of:
providing a flexible gas permeable container, the container comprising:
a supporting container comprising a first side wall connected to a portion of an opposing second side wall along a peripheral seal to define a containment area, each side wall having an interior surface, the first side wall being constructed from a gas permeable material selected from the group consisting of: polymeric material, paper, and fabric, the first side wall having a gas permeability sufficient to permit cellular respiration, and the second side wall being constructed from a material selected from the group consisting of: polymeric material, paper, fabric, and foil;
forming a fibrin matrix layer on a portion of the interior surface of at least one of the side walls of the supporting container; and
introducing a cell line into the containment area of the container to allow the cells to attach to the fibrin matrix.

55. The method of claim 54, wherein the gas permeable material is selected from the group consisting of: ethylene vinyl acetate copolymers, polyolefins, polyamides, polyesters, styrene and hydrocarbon copolymers fluorocarbon elastomers.

56. The method of claim 55, wherein the polymeric material of the first side wall or the second side wall is a multiple-component polymer blend.

57. The method of claim 56, wherein at least one of the components of the multiple-component polymer blend is a styrene and hydrocarbon copolymer.

58. The method of claim 54, wherein the gas permeable material is a monolayer structure.

59. The method of claim 54, wherein the gas permeable material is a multilayer structure.

60. The method of claim 59, wherein the multilayer structure comprises:
a first layer comprising a seventh ethylene vinyl acetate copolymer, the first layer having a first surface and a second surface; and
a second layer adhering to the first surface of the first layer, the second layer comprising an eighth ethylene vinyl acetate copolymer;
wherein the second surface of the first layer forms the interior surface of the supporting container.

61. The method of claim 60, wherein the vinyl acetate content of the ninth vinyl acetate copolymer is greater than 18% by weight of the copolymer.

62. The method of claim 60, wherein the vinyl acetate content of the tenth vinyl acetate copolymer is less than 18% by weight of the copolymer.

63. The method of claim 60, wherein the vinyl acetate content of the ninth vinyl acetate copolymer is about 18% by weight of the copolymer.

64. The method of claim 60, wherein the vinyl acetate content of the tenth vinyl acetate copolymer is about 9% by weight of the copolymer.

65. The method of claim 60, wherein the multilayer structure comprises:
a first layer comprising polystyrene having a thickness within the range of 0.0001 inches to about 0.0010 inches; and
a second layer adhering to the first layer, the second layer comprising a polymeric material selected from the group consisting of ethylene vinyl acetate copolymers, polyolefins, polyamides, polyesters, styrene and hydrocarbon copolymers, fluorocarbon elastomers, the second layer having a thickness within the range of 0.004 inches to about 0.025 inches.

66. The method of claim 65, wherein the polymeric material of the second layer is a multi-component polymer blend.

67. The method of claim 66, wherein at least one of the components of the multi-component polymer blend is a styrene and hydrocarbon copolymer.

68. The method of claim 65, wherein the fibrin matrix is positioned on a portion of the polystyrene layer covering substantially an entire surface of the polystyrene layer.

69. The method of claim 54, wherein the second side wall of the supporting container is constructed from a gas permeable material selected from the group consisting of: polymeric materials, paper, and fabric.

70. The method of claim 69, wherein the polymeric material of the second side wall is selected from the group consisting of: ethylene vinyl acetate copolymers, polyolefins, polyamides, polyesters, styrene and hydrocarbon copolymers, and fluorocarbon elastomers.

71. The method of claim 70, wherein at least one of the components of the multi-component polymer blend is a styrene and hydrocarbon copolymer.

72. The method of claim 69, wherein the gas permeable material is a monolayer structure.

73. The method of claim 69, wherein the gas permeable material is a multilayer structure.

74. The method of claim 73, wherein the multilayer structure comprises:
a first layer comprising a ninth ethylene vinyl acetate copolymer with a vinyl acetate content of greater than 18% by weight of the copolymer, the first layer having a first surface and a second surface; and
a second layer adhering to the first surface of the first layer, the second layer comprising a tenth ethylene vinyl acetate copolymer with a vinyl acetate content of less than 18% by weight of the copolymer;
wherein the second surface of the first layer forms the inner surface of the supporting container.

75. The method of claim 74, wherein the a vinyl acetate content of the ninth vinyl acetate copolymer in the first layer is about 18% by weight of the copolymer.

76. The method of claim 74, wherein the a vinyl acetate content of the tenth vinyl acetate copolymer in the second layer is about 9% by weight of the copolymer.

77. The method of claim 73, wherein the multilayer structure comprises:
a first layer comprising polystyrene having a thickness within the range of 0.0001 inches to about 0.0010 inches; and
a second layer adhering to the first layer, the second layer comprising a polymeric material selected from the group consisting of ethylene vinyl acetate copolymers, polyolefins, polyamides, polyesters, styrene and hydrocarbon copolymers, the second layer having a thickness within the range of 0.004 inches to about 0.025 inches.

78. The method of claim 77, wherein the polymeric material of the second layer is a multi-component polymer blend.

79. The method of claim 78, wherein at least one of the components of the multi-component polymer blend is a styrene and hydrocarbon copolymer.

80. The method of claim 54, wherein the container having an oxygen permeability of from about 9 to about 15 Barrers, a carbon dioxide permeability of from about 40 to about 80 Barrers, a nitrogen permeability of from about 10 to about 100 Barrers, and a water vapor transmission rate of less than about 20 (g mil/100 in²/day).

81. The method of claim 54, wherein the first side wall and the second side wall having a flexural modulus of from about 10,000 to about 30,000 psi as measured according to ASTM D-790.

82. The method of claim 54, wherein at least a portion of the container is optically clear.

83. The method of claim 54, wherein a substantial portion of the container is optically clear.

84. The method of claim 54, wherein the container is radiation sterilizable.

85. The method of claim 54, wherein the container further comprising at least one port providing access to the containment area.

86. The method of claim 54, wherein the fibrin matrix extends over substantially an entire surface of the interior surface of at least one of the side walls.

87. The method of claim 54, wherein the fibrin matrix is on at least a portion of the interior surface of each of the side walls.

88. The method of claim 54, wherein the fibrin matrix is three-dimensional with pore sizes of from about 0.5 to about 5.0 um in diameter.

89. The method of claim 54, wherein the fibrin matrix is formed by cross-linking fibrin or fibrinogen.

90. The method of claim 54, wherein the fibrin matrix is prepared by mixing a first solution comprising fibrinogen and factor XIII with a second solution comprising thrombin and calcium to form the fibrin matrix.

91. The method of claim 90, wherein the fibrinogen is derived from mammalian plasma.

92. The method of claim 91, wherein the mammalian plasma is human plasma.

93. The method of claim 90, wherein the fibrinogen is a recombinant fibrinogen.

94. The method of claim 90, wherein the factor XIII is derived from mammalian plasma.

95. The method of claim 94, wherein the mammalian plasma is human plasma.

96. The method of claim 90, wherein the factor XIII is a recombinant factor XIII.

97. The method of claim 90, wherein the thrombin is derived from mammalian plasma.

98. The method of claim 97, wherein the mammalian plasma is selected from the group consisting of bovine plasma and human plasma.

99. The method of claim 90, wherein the thrombin is a recombinant thrombin.

100. The method of claim 90, wherein the concentration of fibrinogen in the mixture in the first solution is from about 2.0 to about 20 mg/mL, the concentration of the factor XIII in the first solution is from about 10 to about 40 IU/mL, the concentration of the thrombin in the second solution is from about 2.5 IU/mL to about 50 IU/mL, and the concentration of the calcium in the second solution is from about 40 to about 100 mmoles/mL. Approximately 0.5-1.0 mLs of the first solution is mixed with 0.5-1.0 mLs of the second solution to form a fibrin-forming mixture. The polymerization reaction takes place at room temperature in 1-5 minutes and is complete in about 5-15 minutes at 37° C. The fibrin matrix formed in this embodiment has a pore size of about 0.5-5.0 μm in diameter.

101. The method of claim 54, further comprising the step of introducing one or more factors to the containment area to enhance cell attachment and proliferation.

102. The method of claim 101, wherein the factor to enhance cell attachment and proliferation comprises serum proteins.

103. The method of claim 102, wherein the factor to enhance cell attachment and proliferation is fetal calf serum.

104. The method of claim 54, wherein the cell line is selected from the group consisting of human islets of Langerhans and insulin-producing endocrine cells.

105. The method of claim 54, wherein the cell line comprising progenitor cells.

106. The method of claim 104, wherein the progenitor cells are selected from the group consisting of pancreatic duct progenitor cells and islet-derived progenitor cells.

107. The method of claim 54, wherein the method of forming the fibrin matrix in the supporting container comprising the steps of:
providing a first solution comprising fibrinogen and factor XIII;
providing a second solution comprising thrombin and calcium;
mixing the first solution and the second solution thoroughly and rapidly to form a mixture;
introducing the mixture into the container and coating the mixture on a portion of the interior surface of at least one of the side walls of the supporting container; and
allowing the mixture to form a three-dimensional network of fibrin matrix on the inner surface of the side wall of the container.

108. The method of claim 107, wherein the fibrinogen is derived from mammalian plasma.

109. The method of claim 108, wherein the mammalian plasma is human plasma.

110. The method of claim 108, wherein the fibrinogen is a recombinant fibrinogen.

111. The method of claim 108, wherein the factor XIII is derived from mammalian plasma.

112. The method of claim 111, wherein the mammalian plasma is human plasma.

113. The method of claim 106, wherein the factor XIII is a recombinant factor XIII.

114. The method of claim 106, wherein the thrombin is derived from mammalian plasma.

115. The method of claim 106, wherein the mammalian plasma is selected from the group consisting of bovine plasma and human plasma.

116. The method of claim 107, wherein the thrombin is a recombinant thrombin.

117. The method of claim 107, wherein the concentration of fibrinogen in the mixture in the first solution is from about 2.0 to about 20 mg/mL, the concentration of the factor XIII in the first solution is from about 10 to about 40 IU/mL, the concentration of the thrombin in the second solution is from about 2.5 IU/mL to about 50 IU/mL, and the concentration of the calcium in the second solution is from about 40 to about 100 mmoles/mL. Approximately 0.5-1.0 mLs of the first solution is mixed with 0.5-1.0 mLs of the second solution to form a fibrin-forming mixture. The polymerization reaction takes place at room temperature in 1-5 minutes and is complete in about 5-15 minutes at 37° C. The fibrin matrix formed in this embodiment has a pore size of about 0.5-5.0 μm in diameter.

118. The method of claim 107, wherein the step of introducing the mixture to the containment area of the container is via an access port on the body of the container.

119. The method of claim 107 wherein the step of introducing the mixture to the containment area of the flexible gas permeable container is by spraying the mixture onto the interior surface of the side wall of the container.

120. The method of claim 54, wherein the method of forming the fibrin matrix in the supporting container comprises the steps of:
providing a dry fibrin matrix;
introducing the dry fibrin matrix into the supporting container; and
rehydrating the dry fibrin matrix in the supporting container.
